(19) United States
(12) Patent Application Publication
(10) Pub. No.: US 2013/0046174 A1
(43) Pub. Date: Feb. 21, 2013

(54) DEVICE FOR PLACING A MARKER INTO A HUMAN SUBJECT FOR THE PURPOSE OF TISSUE BIOPSY

(71) Applicants: ROBERT E. FISCHELL, DAYTON, MD (US); DAVID MULLEN, GREENWICH, CT (US); MARK ZYZELEWSKI, KALAMAZOO, MI (US)

(72) Inventors: ROBERT E. FISCHELL, DAYTON, MD (US); DAVID MULLEN, GREENWICH, CT (US); MARK ZYZELEWSKI, KALAMAZOO, MI (US)

(73) Assignee: MAGNEMARK, LLC, DAYTON, MD (US)

(21) Appl. No.: 13/209,487

(22) Filed: Aug. 15, 2011

(51) Int. Cl. A61B 6/00

(52) U.S. Cl. 600/431

(57) ABSTRACT

The present invention is a marker delivery device designed to deliver an elongated magnet to mark the center of tissue that is suspected of being cancerous. This device has an elongated magnet situated within a hollow needle with the magnet's distal end placed at the distal end of the needle. The operator first places the tip of the needle at the center of the lesion. By pressing an activate button on the injector device, the needle is advanced by half the length of the magnet and then pulled back the full length of the magnet while a rod within the needle that is in contact with the proximal end of the magnet prevents the magnet from coming back when the needle goes back. Thus the magnet is deployed with its center at the center of the lesion that is suspected of being cancerous.

### FIELD OF USE

This invention is in the field of devices for marking of tissue in a human subject that is at risk for being cancerous tissue.

### BACKGROUND OF THE INVENTION

An important function of an interventional radiologist is to mark tissue in a human subject that is thought to be cancerous so that a surgeon can subsequently remove that tissue and then have it examined by a pathologist. This has been best described in the diagnosis and treatment of breast cancer which is the most common cancer that occurs in woman but is also of increasing importance in other organs, especially the lung. By far the most common way in which this is now accomplished is by positioning a temporary marker, most frequently constructed of a metal anchor at the end of a wire inserted through a needle that has been accurately positioned by image guidance prior to the release of the marker. (See, Frank H. A., Hall F. M., Steer M. L., Preoperative Localization of Nonpalpable Breast Lesions Demonstrated by Mammography; New England Journal of Medicine, 1976; 296:259-260). Many metal devices to accomplish breast marking or localization have been devised (e.g., U.S. Pat. Nos. 4,799,495; 5,011,473; 5,057,085; 5,083,570; 5,127,916; 5,158,084; 5,221,269; 5,234,426; 5,409,004; 5,556,410; 6,053,925; and 6,544,269). Because the anchoring device is typically connected to a wire that protrudes through the skin, it must be promptly removed. The need for immediate preoperative localization creates logistical problems for radiology departments and operating room personnel. Any system that could eliminate the need for prompt surgery after localization of the suspected tissue volume would be an advance in this field. An improved means to mark suspected tissue would be of considerable value if it does not require the patient to remain in the hospital until an operating room becomes available so that the surgical procedure can be accomplished.

In U.S. Pat. No. 6,698,433, D. N. Krag describes several means to localize a suspected tissue volume. These methods include the placement of tiny magnets in the breast to be detected by a magnetometer. Specifically, FIG. 2b of the Krag patent shows a tiny magnet which has a diameter that is actually greater the length of the magnet between the magnetic poles. That is, the ratio of the distance between the poles compared to the magnet diameter is less than 1.0. Such tiny magnets would have an extremely small field at any reasonable distance outside of a human breast because the close proximity of the north and south poles results in cancellation of the magnet's external field. Such magnets placed in a suspected tissue volume would not be at all detectable using a magnetometer probe outside of the breast. This is in contradistinction to a single elongated magnet with a length that would be at least five times its diameter so that its magnetic field would be readily detectable outside of the breast.

In U.S. Pat. No. 5,622,169 Golden et al. disclose an apparatus and method to locate a medical tube in the body of a patient. Golden is primarily concerned with the description of a magnetic field detector, but also teaches fixing an elongated magnet to the end of a medical tube wherein the magnet is a cylinder that has a width of 0.10 inches and has a length of 0.25 to 0.50 inches (see column 8, line 66). However, Golden teaches only a method for placing the magnet into the body of a patient by first attaching the magnet to the end of a medical tube and then inserting the tube with the magnet into a preexisting body cavity. This technique would not be viable for percutaneous placement of a magnet into an organ in the body of a patient where there is no pre existing body cavity.

In U.S. Pat. No. 6,173,715 Sinanan et al teach an elongated cylindrical magnetic marker that can be inserted into the wall of the bowel by using an insertion tool that is usable with an endoscope (see Abstract). The teaching of Sinanan (like that of Golden) requires that the magnet be inserted into a pre-existing body cavity and does not in any way describe a means to percutaneously insert and then secure at a fixed position a magnet into human tissue for subsequent detection by a magnetic field detector. Further, although Sinanan describes an eccentric absorbable anchor to hold the magnet in place by anchoring the magnet to the wall of the bowel, he in no way describes a means to precisely control or maintain the location where the magnet deploys with respect to a lesion.

The magnet markers of Golden or Sinanan only reveal the general location of the marker at a substantial distance from the detection apparatus, which is to say that while the magnet in the tip of the tube (Golden) or bowel wall (Sinanan) is inside the body (i.e., the stomach) the detection apparatus is outside the body (Golden) or outside the bowel (Sinanan). In fact, Golden teaches that his detector is designed to operate at a range of “several centimeters to several decimeters” (column 2, lines 33-36 and column 3, lines 27-29) and teaches nothing at all about the special problems associated with the detection of a magnet at extremely close range as would be required for location within a female breast.

For a magnetic field detecting apparatus to function at any reasonable distance, the magnetic marker must have reasonably high field strength. In order to comfortably insert a magnet percutaneously, it must have a reasonably small outer diameter. Thus, a magnet optimal for both percutaneous insertion and subsequent detection will have an outer diameter of about 1 mm and a length of approximately 20 mm. Since it is the case that most suspicious lesions are small in comparison to the length of an elongated magnet which is long enough to be detectable by a magnetic field detector at any reasonable range, it is critically important that the exact location of the lesion with respect to the magnet is reliably and precisely controlled. The problem of detecting a magnet at close range is not trivial. Since the magnitude of a magnetic field is greatest at the north and south poles, a magnetic field detector such as taught by Golden that relies primarily on detecting the absolute magnitude of a magnetic field will return a higher detection signal when approaching the north or south pole than when approaching the center of an elongated magnet; a problem that becomes exponentially more pronounced when at close range. This problem has the effect of producing significant uncertainty as to the exact location of the marked tissue when a magnetic gradiometer measuring the absolute magnitude of magnetic field strength approaches the near field of the magnet. However, if the lesion could be marked by the exact center of the magnet (that is to say the precise midpoint between the north and the south poles of an elongated cylindrical magnet) this problem may be addressed by the full analysis of the three dimensional vectors of the magnetic field.

In U.S. Pat. No. 7,577,473 Davis et al and in U.S. Pat. Nos. 7,569,065 and 6,575,991 Chesbrough et al describe an apparatus for subcutaneous placement of a non-magnetic imaging marker. This includes a sharpened cannula that can be used to puncture the breast percutaneously and deposit a very small non-magnetic marker into the center of a lesion by first positioning the tip of the cannula in the center of the lesion and then pushing the marker out of the cannula with a sliding stylet. Although this results in the approximate center of the lesion being marked by a marker which is small in comparison to the diameter of the marked lesion (see Davis et al FIGS. 9 and 11), this method would not work for inserting an elongated cylindrical magnet so that the center of the lesion is marked by the center of the magnet. This is because a marker of substantially greater length than the diameter of a small lesion when deployed in this manner would have its center far away from the center of the suspected tissue. Thus the surgeon performing the biopsy would not know where the center of the lesion would be located.

In US Patent Application 2004/0122312, Chesbrough et al describe an apparatus and method for implanting a localizing wire into a breast lesion, teaching a sharpened cannula containing a wire that looks much like a standard Kopans breast localization wire when shown deployed in the lesion (see FIG. 9). The wire is deployed by pushing a button that causes the cannula to be withdrawn by a spring, thus depositing the distal end of the localization wire into the tissue mass (see abstract and claim 1). Chesbrough teaches that the automatic implanting is more accurate than manual deployment of a marker since the user will be less likely to move the apparatus relative to the mass during marker deployment (paragraph [0104]) and further teaches that automatically implanting a marker is improved by requiring only the placement and visualization of the needle tip into the center of the target prior to actuation of the automatic deployment mechanism (paragraph [0105]). What Chesbrough does not does not teach or anticipate is an automated means of inserting an elongated cylindrical marker (whether magnetized, unmagnetized, fully implantable or attached to a localization wire) such that the exact center of the cylindrical marker is automatically and reproducibly deployed and anchored at the center of the target lesion.

In U.S. Pat. No. 4,699,154 Lindgren discloses a tissue sampling device in which an automated spring loaded mechanism drives the tissue sampling needle in a two phase fashion. Lindgren teaches a means to sample tissue by first manually positioning the needle tip, then activating the automatic advancement of the inner tissue sampling needle containing a hollow 3b immediately followed by the advancement of the outer needle 2 over the hollow 3b (see FIGS. 1a, 1b and 1c). Lindgren does not show the position of the hollow 3b with respect to the tissue being sampled, but in U.S. Pat. No. 5,538,010 Darr et al show that the biopsy chamber of a device representative of the prior art may be expected to be centered in the center of a lesion 18 before the tissue is removed (see FIGS. 1a, 1b and 1c). Neither Lindgren nor Dan teach anything of the placement of a biopsy site marker with such a tissue sampling device, but in U.S. Pat. No. 6,056,700 Burney et al describe a biopsy marker and method of use that deploys a marker into a biopsy site immediately after a biopsy sample has been obtained. Although Burney teaches an elongated cylindrical marker that is roughly centered within the biopsy site (and therefore roughly centered on the target lesion—see FIGS. 4 and 6), the teaching of Burney has important limitation when considering the special needs of an elongated cylindrical magnetic marker. Specifically, the marker of Burney is deployed from within the sampling cavity of a biopsy needle and is therefore limited in outer diameter to the size of the sampling cavity. This limitation therefore limits the outer diameter of the marker to the diameter of the sampling cavity. As shown in FIG. 1 of Burney, the outer diameter of the marker 20 is of necessity very significantly less than the inner diameter of the outer needle 12. Although this may suit the teaching of Burney since his concern is only that the marker be imaged at some later date (i.e. by mammography), this limitation would have the effect of greatly reducing the magnetic field strength of an elongated cylindrical magnetic marker that might be deployed according to the method of Burney. Since the field strength of a cylindrical magnet of any given length increases as the square of the radius of the cylinder, even a small increase in the outer diameter of a magnetic marker will be of great importance in the success of a magnetic marking procedure.

Thus what is needed to advance the field, but is not taught by the prior art, is a means and method for the percutaneous placement of an elongated magnet into the human body such that the center of the magnet is automatically, precisely and reproducibly secured within the center of a suspicious lesion so that a surgeon may later detect the location of such a lesion by the use of a magnet locating system. Advantageously, the device for such magnet insertion should be fully automated so that the operator must only position and visualize the tip of the insertion device at the center of the target before actuating the deployment mechanism. The insertion device should further be designed to maximize the outer diameter of the magnetic marker for any given outer diameter of the insertion device so as to maximize the magnetic field of the marker. Advantageously, the magnet should be fully implanted into the human body without attachment to any wire, and left in place at the approximate center of the suspected tissue for later surgical excision of the magnet with the tissue that is suspected of being cancerous.

### SUMMARY OF THE INVENTION

The present invention is a marker delivery device whose goal is to deliver a marker that is an elongated permanent magnet. For the purposes of this specification, the marker delivery device that injects the magnet into the suspected tissue will be called a “MagneJector.” The goal of the MagneJector is to place the center of an elongated magnet at the approximate center of the tissue that is suspected of being cancerous. This is accomplished by placing the magnet within a distal section of a hollow needle that has a pointed distal tip with the distal end of the magnet being generally situated at the distal tip of the needle. Within the hollow needle is an elongated push rod whose distal end is at the proximal end of the magnet.

The marker delivery device (the MagneJector) is first used to place the distal tip of the needle at the approximate center of the suspected tissue. Since it is easiest for the radiologist to observe the distal tip of the marker delivery device, it is most desirable to place that needle tip at the center of the suspected tissue. The radiologist can use various well known means to observe the position of the tip of the needle during needle positioning for example by ultrasound, mammography, stereotactic mammography, computerized tomographic scanning, fluoroscopy, or any other means of imaging known to those versed in the art.

In a first embodiment, the MagneJector may either be held in the operators hand and the needle inserted under real time ultrasound guidance or mounted on a rail and positioned using stereotactic mammographic guidance. Once the tip of the needle is placed at the approximate center of the suspected tissue and its position is confirmed, the radiologist actuates the MagneJector to first advance the needle, rod and magnet by a distance that is half the length of the magnet and then the MagneJector pulls the needle back by the length of the magnet while the rod within the needle holds the magnet in a fixed position. The retraction of the needle by the entire length of the magnet while the rod holds the magnet in its place causes the magnet to be released from inside the needle and the magnet is then accurately placed into the breast tissue with the center of the magnet at the position that was previously occupied by the tip of the needle. Thus, the magnet is released with the center of the magnet being placed at the approximate center of the suspected tissue. That centering of the magnet at the center of the suspected tissue in the female breast is an important objective of the present invention. A center anchor device can be used with the magnet to keep it in place within the breast tissue until it is removed by the surgeon. After the magnet is placed, a special type of magnetic gradiometer can then be used by the surgeon to locate the center of the magnet to guide the removal of the suspected tissue.

An alternative embodiment of the present invention utilizes a separate needle assembly consisting of an elongated magnet placed inside of a needle that can be inserted into breast tissue without initially being attached to the MagneJector delivery device. During this insertion, the magnet is held in place by friction from its center anchors and a cylindrical rod placed inside the needle, the cylindrical rod having a Luer fitting at its proximal end that is removably attached to a Luer fitting at the proximal end of the needle. This embodiment will prove most useful in applications wherein the needle must be unsupported during certain phases of the imaging operation such as during mammographic or computed tomographic guided insertions. In this embodiment, the needle may be manually inserted, released from manual support, its position checked by imaging and then repositioned as needed prior to the attachment of a MagneJector delivery device. Once the tip of the needle is placed at the approximate center of the suspected tissue and its position is confirmed, the cylindrical rod can be removed from its Luer fitting and withdrawn from the needle and a MagneJector delivery device can be attached to the Luer fitting, allowing the injection of the magnet just as described above.

Thus one object of the present invention is place an elongated permanent magnet with its center at the approximate center of tissue within a human subject that is suspected of being cancerous.

Another object of this invention is to use a marker delivery device that first advances a distance equal to half the length of an elongated magnet and then the needle of the MagneJector is pulled back by the entire length of that magnet so as to release the magnet into the tissue of the human subject with the center of the magnet placed at the approximate center of the tissue to be excised.

Still another object of this invention is to utilize a magnet that has a center anchor so as to assure that the magnet remains in a fixed position within the suspected tissue with the center of the magnet being at the approximate center of the tissue that is suspected of being cancerous.

Still another object of this invention is to utilize a magnet that has a center anchor that exerts a frictional force against the inner surface of the needle into which the magnet is placed prior to insertion of the magnet into the suspected tissue so as to prevent the inadvertent discharge of the magnet from the open end of the needle.

A most important object of the present invention is have a means to place a marker at the approximate center of suspected tissue without having any part of the marker protrude through the skin so that the surgical excision of that marker could be accomplished at some later time that is convenient for both the patient and the surgeon who would remove that marker.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1A is a top view of the MagneJector prior to release of the elongated magnet.

FIG. 1B is an angled view of the MagneJector of FIG. 1A.

FIG. 2A is a top view of the MagneJector showing the position of the actuation springs and the distal end of the sharp needle during and after injection of the elongated magnet.

FIG. 2B is an angled view of the MagneJector showing the position of the actuation springs and the distal end of the sharp needle during and after injection of the elongated magnet.

FIG. 3A is a cross section of a distal section of the needle showing the position of the elongated magnet and the push rod prior to injection of the magnet into the tissue of the human subject.

FIG. 3B is a cross section of a distal section of the needle showing the position of the elongated magnet and the push rod as the needle is advanced into the tissue of the human subject.

FIG. 3C is a cross section of a distal section of the needle showing the position of the elongated magnet and the push rod after injection of the magnet into the tissue of the human subject having achieved the goal of placing the center of the magnet at the center of the suspected tissue.

FIG. 4A is a cross section of a distal section of the needle of the MagneJector showing an elongated magnet with a center anchor contained within the needle of the MagneJector.

FIG. 4B is a cross section of an elongated magnet tissue marker showing the center anchor in its deployed configuration.

FIG. 5 is a longitudinal cross section of an alternative embodiment of the present invention which is a needle assembly designed for removable attachment to a MagneJector, which needle assembly is designed for placement into breast tissue without first being attached to MagneJector itself.

FIG. 6A is a top view of the MagneJector for the second embodiment of the present invention, this version of the MagneJector having a solid cylindrical push rod attached to a sliding Luer fitting mounted at the distal end of the MagneJector.

FIG. 6B is an angled view of the MagneJector of FIG. 6A.

FIG. 7A is a top view of the MagneJector about to be attached to the needle assembly in which the push rod with a proximal Luer fitting has been previously removed from the hollow needle.

FIG. 7B is an angled view of the MagneJector of FIG. 7A.

FIG. 8A is a top view of the MagneJector showing the needle with magnet enclosed being advanced onto the push rod that extends from the sliding Luer fitting of the MagneJector.

FIG. 8B is an angled view of the MagneJector plus needle as shown in FIG. 8A.

FIG. 9A is a top view of the MagneJector showing the attachment of the Luer fitting on the needle to the sliding Luer fitting that is part of the MagneJector.

FIG. 9B is an angled view of the MagneJector plus needle as shown in FIG. 9A.

FIG. 10A is a top view of the MagneJector plus needle with the sliding Luer fitting being advanced in a forward direction for a distance L s as to place the magnet within the needle at its appropriate position which is approximately centered on the tissue that is suspected of being cancerous.

FIG. 10B is an angled view of the MagneJector plus needle as shown in FIG. 10A.

FIG. 11A is a top view of the MagneJector plus needle with the sliding Luer fitting pulled back a distance 2L so as to release the magnet with its center at the center of the breast lesion; and,

FIG. 11B is a perspective view of the MagneJector plus needle as shown in FIG. 11A.

### DETAILED DESCRIPTION OF THE INVENTION

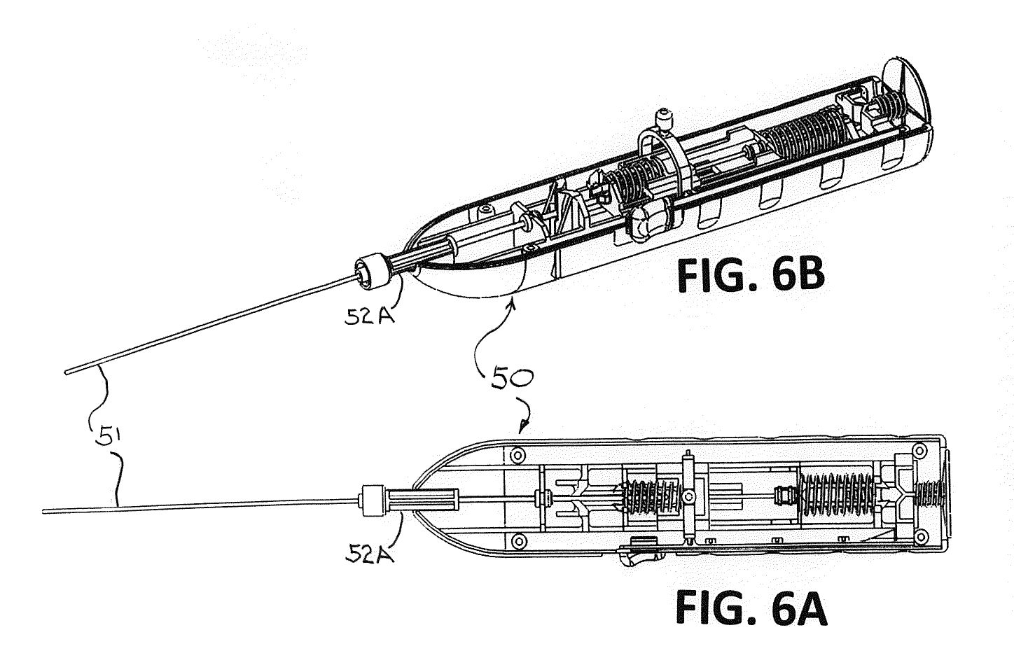

The present invention is a marker delivery device whose goal is to place an elongated permanent magnet with its center at the approximate center of human tissue that is thought to be cancerous. For the purposes of the specification of this invention, the marker device will be called a MagneJector 10 which has an initial configuration referred to as MagneJector 10A and a final configuration noted as MagneJector 10B. For FIGS. 6A through 11B inclusive, the device to deliver the magnet into the breast tissue will be referred to as MagneJector 50.

The goal of the MagneJector 10 or the MagneJector 50 is to first place the tip of the delivery needle at the center of the tissue that is to be excised by a surgeon. Of all the sections of the needle that could be used for this purpose, it is most logical for the radiologist to observe the tip of the needle to be at the center of the tissue that is to be excised. Then the MagneJector is used to inject the elongated magnet so that its center is centered at the place where the needle tip was initially placed. To accomplish this goal, the MagneJector first advances a distance L and then (without an additional action by the radiologist) retracts a distance 2L. This action achieves the goal of placing the center of the magnet at the position where the needle tip was placed which is at the approximate center of the tissue to be excised.

Although the following description describes placement of the magnet within suspected breast tissue, it should be understood that the present invention could be used for placement of the elongated permanent magnet into any other tissue of a human subject, such as lung tissue, that is suspected of being cancerous.

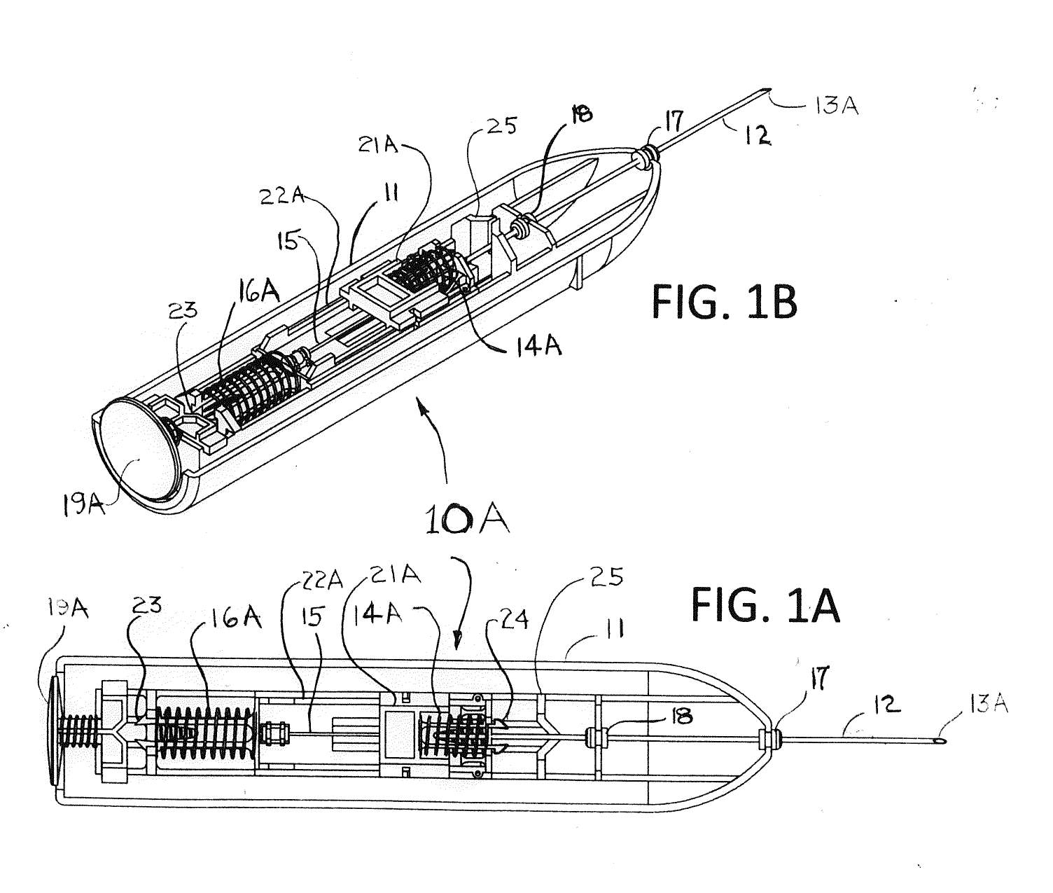

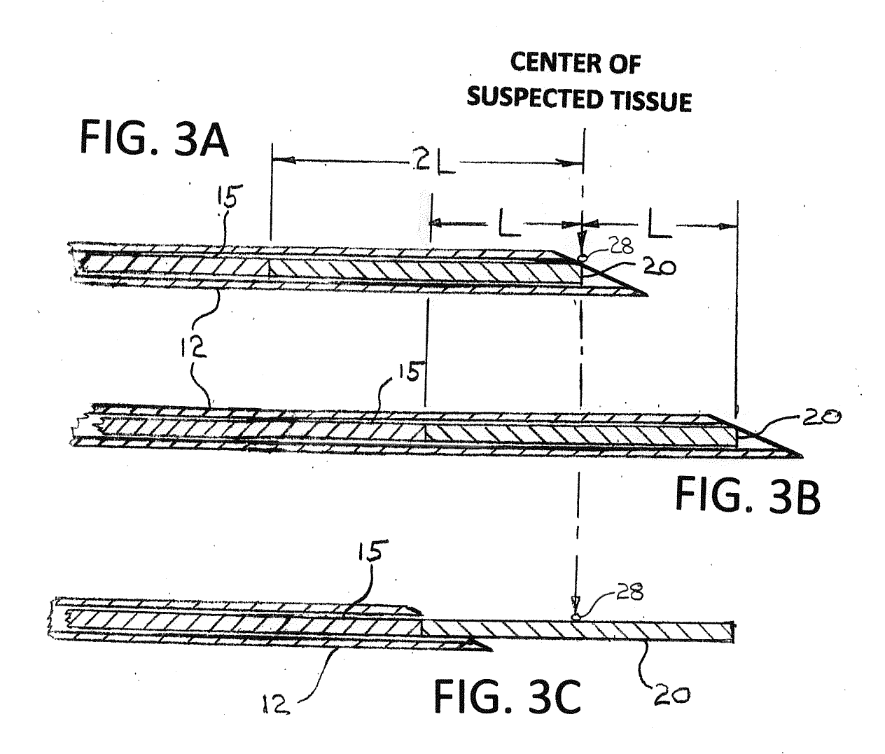

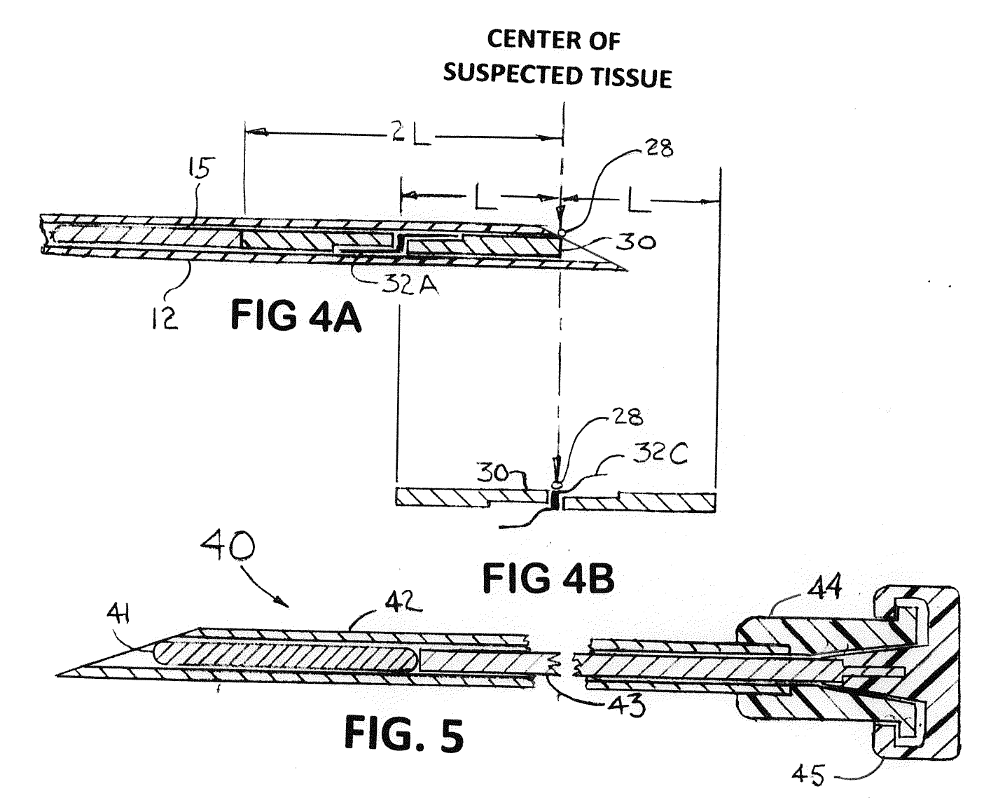

FIGS. 1A, 1B, 3A and 4A show the initial condition of the magnet 20 within the needle 12 of the MagneJector 10A as the needle 12 would be placed into the breast where there is tissue that is suspected of being cancerous. It should be understood that the top of the outer casing 11 of the MagneJector 10 is not shown in any of the figures so as to more clearly show the construction of this device. The MagneJector 10A shown in FIGS. 1A and 1B has an elongated hollow needle 12 with a sharpened distal tip that is initially at a position 13A. Located within a distal section of the needle 12 is the magnet 20 as also seen in FIGS. 3A and 4A. The push rod 15 is also located in the needle 12, and the distal end of the push rod 15 is situated in contact with the proximal end of the magnet 20. The needle 12 is designed to slide through center holes of the short bearings 17 and 18. The needle 12 is fixedly attached to the needle carriage 21A. The push rod 15 is fixedly attached to the rod carriage 22A. The rod carriage 22A can slide within the outer casing 11 of the MagneJector 10A and the needle carriage 21 A can slide upon the rod carriage 22A.

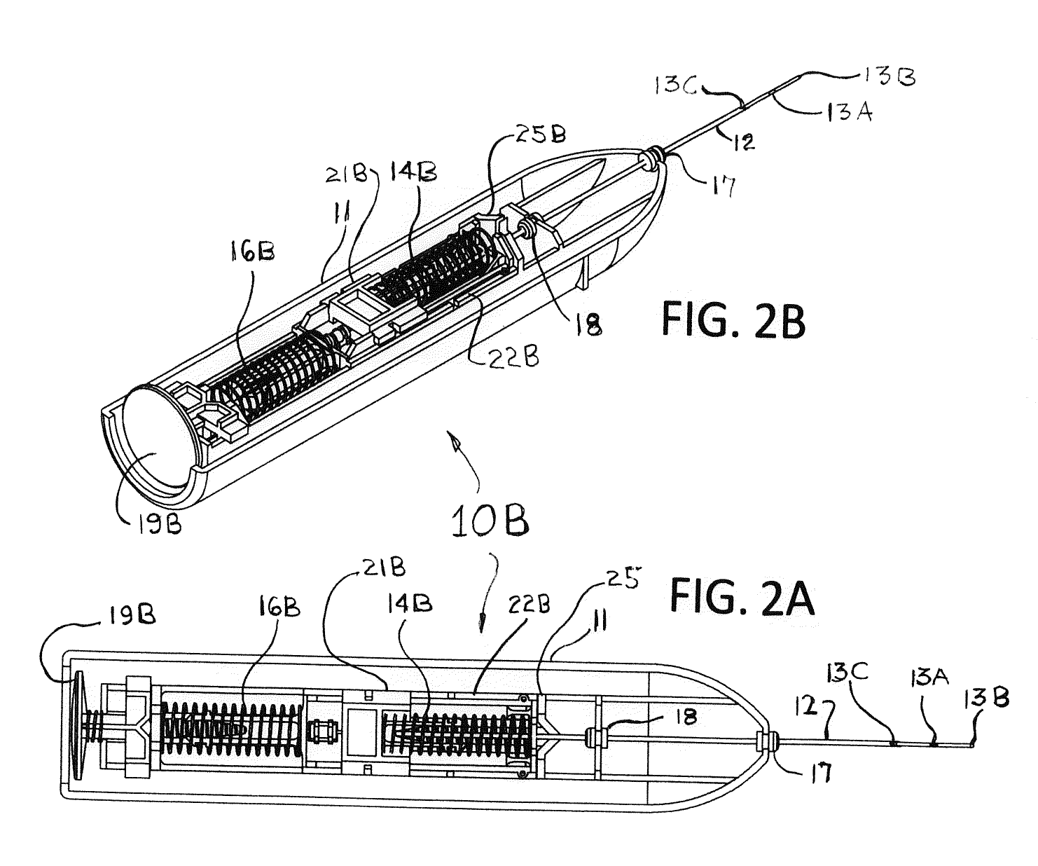

FIGS. 1A and 1B show the device in its pre-actuated position. In this position, the rod carriage 22A is snapped into a fixed slot in the proximal aspect of the outer casing 11 using rod carriage snaps 23. The needle carriage 21A is temporarily attached to the rod carriage 22A using needle carriage snaps 24 snapped into a fixed slot in the distal aspect of the rod carriage 22A. The rod carriage 22A has a spring force applied by the compressed rod spring 16A. When actuated by the actuator button 19A, rod carriage snaps 23 are released and the rod spring 16A will drive the rod carriage 22A together with the needle carriage 21A forward. Due to the fixed attachment of the needle 12 to the needle carriage 21A and the push rod 15 to the rod carriage 22A, the needle 12 and push rod 15 will also both be driven forward in unison as shown in FIGS. 2A, 2B, and 3B.

FIGS. 2A and 2B show the successive positions of the needle 12 after the actuator button 19B is pushed in the forward direction. The tip of the needle 12 is first in the position 13A which position is centered within the tissue in the breast that is suspected of being cancerous. This center position of the lesion is noted by the small circles 28 in FIGS. 3A and 3C and 4A and 4B. Immediately after the tip of the needle 12 reaches position 13B, it is propelled back to its final placement which is position 13C. This movement is accomplished when the rod carriage 22B is driven forward by rod spring 16B. The distal end of the rod carriage 22B is driven forward to the rod carriage stop 25. When the rod carriage 22B hits the rod carriage stop 25, the needle carriage snaps 24 (as shown in FIG. 1A) release the needle carriage 21 B from its temporary attachment to the rod carriage 22B. The needle carriage 21B is then driven backward by the needle spring 14B. This movement of the needle carriage 21B retracts the needle 12 back by at least the full length of the magnet 20. When the needle 12 and the rod 15 reach their final positions, the needle carriage 21B, needle spring 14B, rod carriage 22B, and the rod spring 16B are at their final positions as shown in FIGS. 2A, and 2B with the tip of the needle at position 13C.

FIGS. 3A, 3B and 3C best illustrate the various positions of the needle 12, push rod 15 and magnet 30 each of which have their actions controlled by the MagneJector 10. Specifically, FIG. 3A shows the needle 12, rod 15 and elongated permanent magnet 20 in their initial positions prior to actuation by the actuator button 19A of FIG. 1A. It should be noted that the magnet 20, (which has a length “2L”) is initially placed with its distal end just slightly back from the pointed tip of the needle 12 with the magnet's proximal end in contact with the distal end of the push rod 15.

The initial motion triggered by the actuator button 19A is to move the actuator button to position 19B (as shown in FIG. 1B). This causes the configuration of the needle 12, rod 15 and magnet 30 to be placed as shown in FIG. 3B. This is an initial forward motion that is for a distance “L” which moves the tip of the needle 12 from position 13A (of FIGS. 1A, 1B, 2A and 2B) to position 13B (as shown in FIGS. 2A and 2B) and the rod 15 and magnet 30 are each advanced in a forward direction by a distance “L”. The final positions of the needle 12, rod 15 and magnet 30 are shown in FIG. 3C. In this final position, the tip of the needle 12 is at position 13C as shown in FIGS. 2A and 2B. What is most important is that the elongated permanent magnet 20 has now been released from being within the needle 12 and it is placed with the center of the magnet 20 at the small circle 28 which is located at the approximate center of the breast tissue lesion that is suspected of being cancerous. This placement of the center of the magnet 20 at the center of the suspected tissue is an important objective of the marker delivery device which is the MagneJector 10.

FIG. 4A illustrates a center anchor 32A for a magnet 30 within the needle 12 as it would be situated prior to insertion into breast tissue. When the magnet 30 is released into the center of the suspected tissue as marked by the small circles 28 of FIGS. 4A and 4B, the center anchor 32C will be extended in an outward direction as shown in FIG. 4B. The center anchor 32C allows the magnet 30 to remain in that position where it was delivered by the MagneJector 10. The unique design of the center anchor 32A is such that it fits within the inside diameter of the needle 12 where it performs an important additional function which is to create sufficient friction against the inside surface of the needle 12 so that the magnet does not fall out of the needle 12 prior to insertion of the magnet 30 into the breast tissue. Furthermore, the rod 15 prevents the magnet 30 from sliding back in a proximal direction prior to insertion of the magnet 30 into the breast tissue. Thus, this unique design of an elongated magnet 30 within an elongated needle 12 having a center anchor 32A that provides enough friction to prevent the inadvertent outward motion of the magnet 30 from within the needle 12 with the rod 15 preventing the magnet from sliding back into the needle 12 all work together to provide a reliable means for marking the center of suspected tissue within a human breast. As seen in FIG. 4B, the deployed anchor 32C holds the magnet at the center of the lesion even after decompression of a breast after magnet insertion during mammography has been completed.

An alternative means for accomplishing the placement of an elongated magnet at the center of a breast lesion is shown in FIGS. 6A through 11B inclusive using the needle assembly shown in FIG. 5. This alternative embodiment of the present invention allows the needle assembly containing the magnet to be placed into the breast tissue without having the needle initially attached to the MagneJector. This method for marking of the lesion in the breast is optimum for breast tissue localization when the needle must be unsupported during certain phases of the imaging operation such as during mammographic or computed tomographic guided insertions.

FIG. 5 is a longitudinal cross section of a needle assembly 40 that includes the needle 42 and a magnet 41. The needle 42 has a fixedly attached Luer fitting 44 at its proximal end that is removably attached to the Luer fitting 45 that is fixedly attached to a cylindrical rod 43 that is situated within the needle 42. The needle assembly 40 with the inserted rod 43 is the structure that is used by the radiologist to insert the magnet into the breast when the attachment of the needle to a MagneJector such as the MagneJector 10 of FIG. 1A is precluded because of the method used to detect the lesion in the female breast.

FIGS. 6A and 6B are, respectively, a top view and an angled view of the MagneJector 50 having a sliding Luer fitting 52A with an attached elongated cylindrical rod 51. This is how the MagneJector 50 appears before being attached to the needle assembly 40 of FIG. 5 after that needle assembly 40 has been placed into the breast tissue.

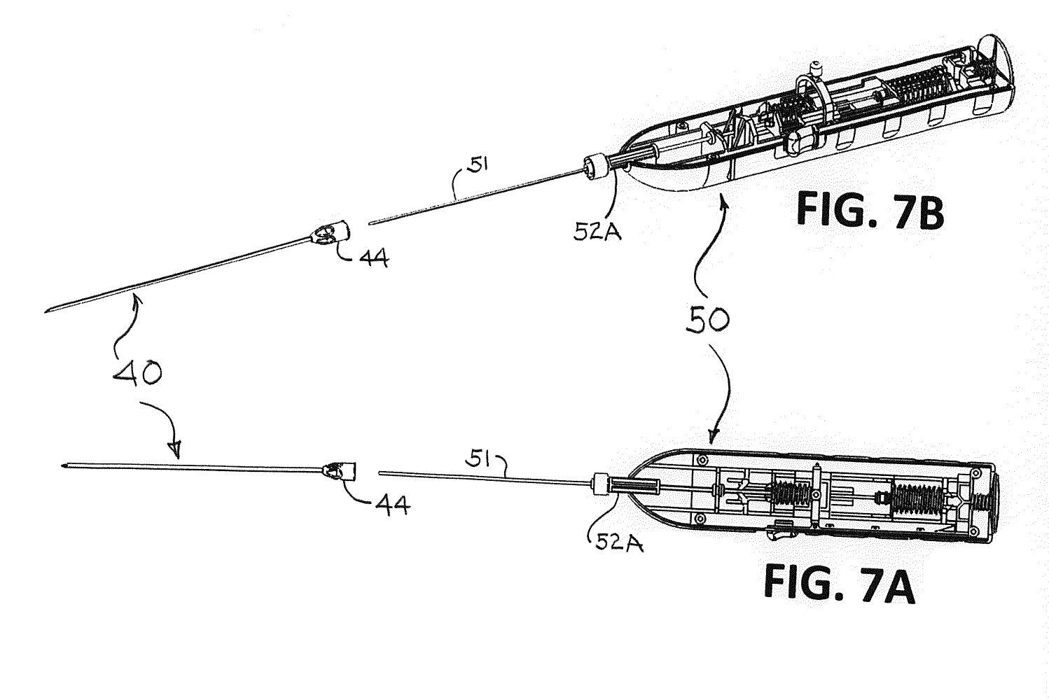

FIGS. 7A and 7B are, respectively, a top view and an angled view of the MagneJector 50 showing the needle assembly 40 with the rod 43 removed about to be slid onto the rod 51 of the MagneJector 50.

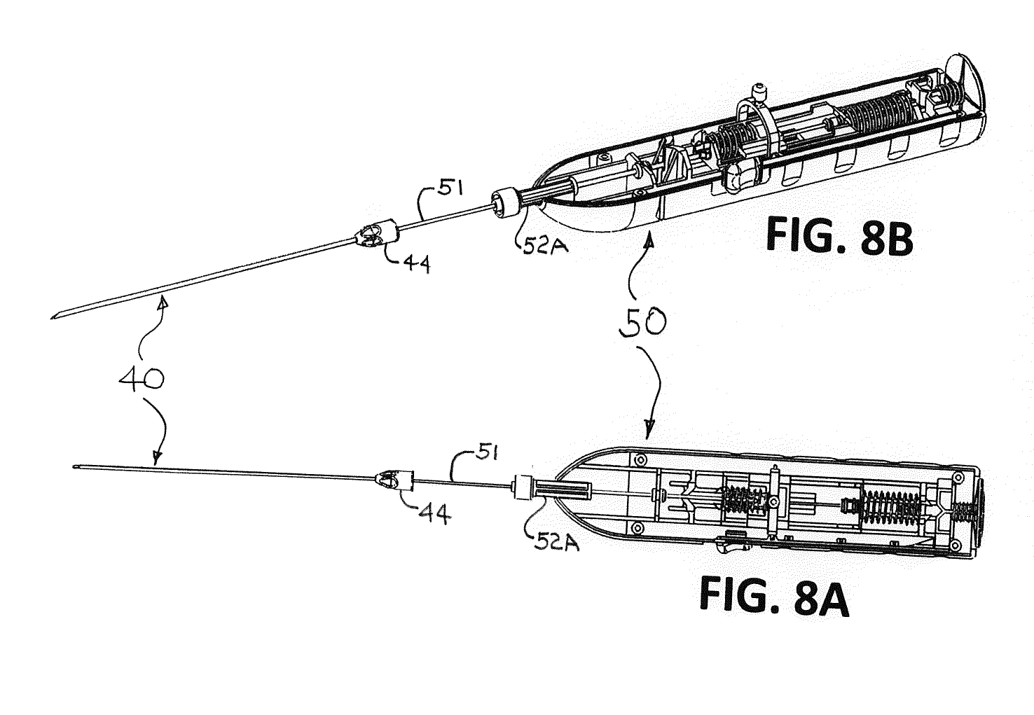

FIGS. 8A and 8B are, respectively, a top view and an angled view of the MagneJector 50 with the needle assembly 40 slid halfway onto the rod 51 of the MagneJector 50.

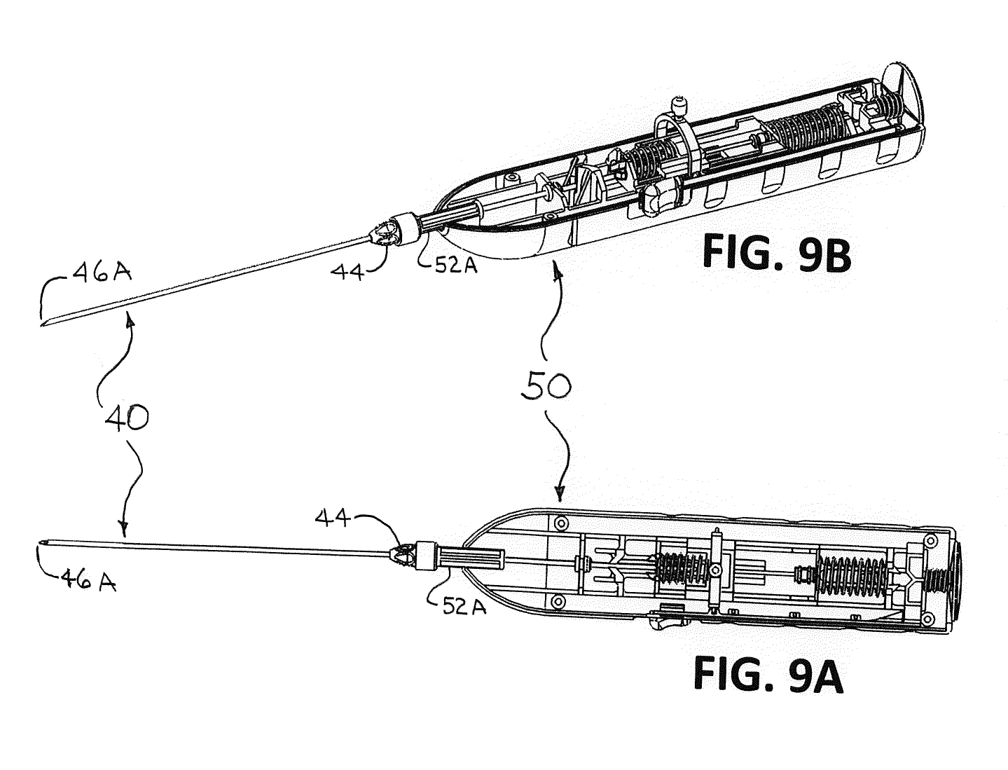

FIGS. 9A and 9B are, respectively, a top view and an angled view of the MagneJector 50 showing the needle assembly 40 being fully slid onto the rod 51 with the needle assembly Luer fitting 44 being attached to the sliding Luer fitting 52A of the MagneJector 50. It should also be noted that the tip of the needle assembly 40 is at a position 46A which is at the center of the tissue that is suspected of being cancerous. It is the goal of this system to place the center of the magnet 41 at that position 46A.

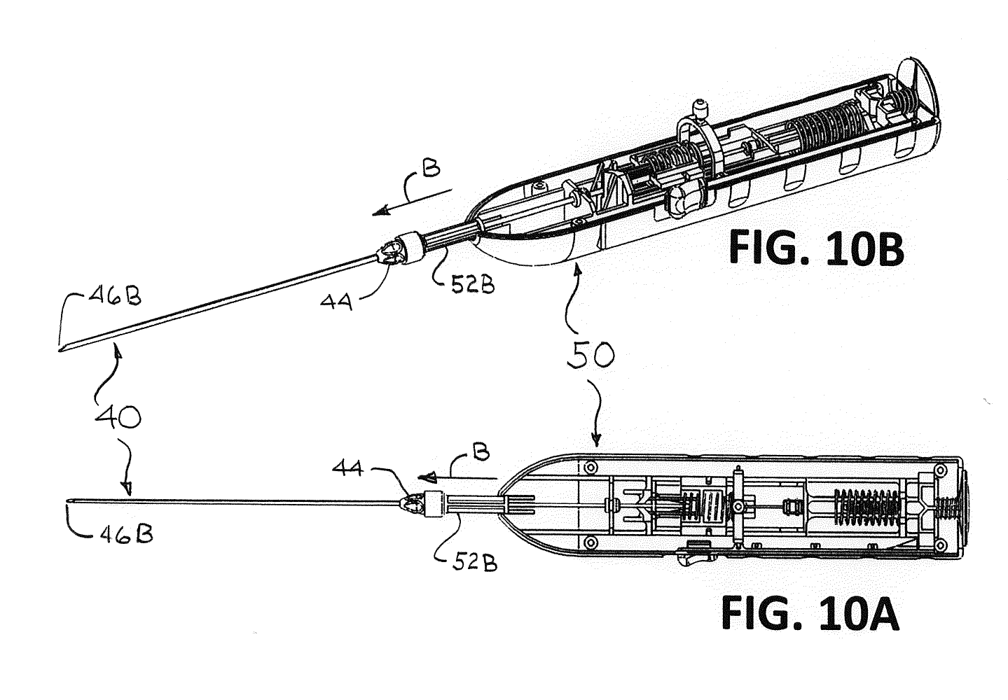

FIGS. 10A and 10B are, respectively, a top view and an angled view of the MagneJector 50 with the sliding Luer fitting 52B having been advanced by a distance equal to half the length of the magnet 41 of FIG. 5. This moves the tip of the needle assembly 40 forward in the direction shown by the arrows B in FIGS. 10A and 10B to the position 46B.

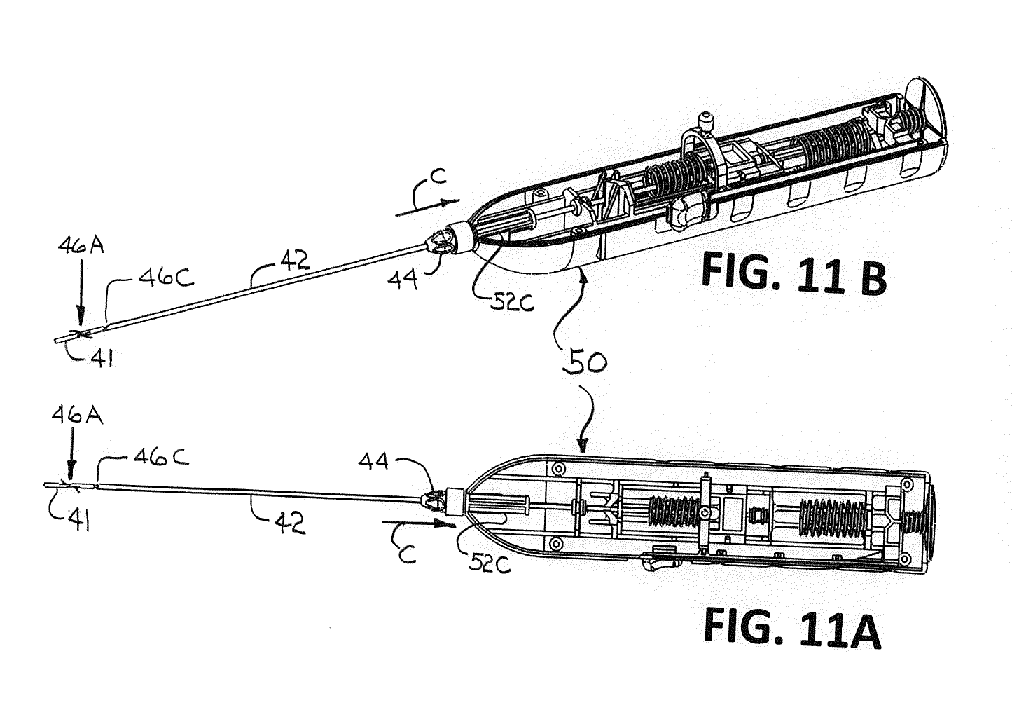

FIGS. 11A and 11B are, respectively, a top view and an angled view of the MagneJector 50 showing the sliding Luer fitting 52C having been pulled back in the direction shown by the arrows C by at least the length of the magnet 41. This motion places the tip of the needle 42 at position 46C. In this position, the magnet 41 is released from within the needle 42 and its center is at the position 46A that was occupied by the needle tip prior to triggering of the motions by the MagneJector 50. Thus the goal of placing the center of the magnet 41 at the position previously set by the tip of the needle 42 has been achieved. It should be noted that the magnet 41 in FIGS. 11A and 11B have center anchors which would be desired to keep the magnet 41 from sliding out of the needle 42 prior to insertion of the needle assembly 40 into the breast and the center anchor also keeps the magnet centered in the breast tissue after any compression of that tissue has been removed.

Although the magnets 20 and 30 are shown with square ends, it is expected that rounded ends may be a more practical design as is shown for the design of the magnet 41 in FIG. 5.

It is well known to make the magnet from a permanent magnet material such as Alnico 5, Vicalloy, Arnochrome 3 or any of the rare Earth magnetic materials that are now on the market. An optimum length for the magnet is about 2 cm and an optimum diameter would be about 1.0 mm. However, it should be understood that magnets of various lengths and diameters could be used with either the MagneJector 10 or MagneJector 50. It should also be understood that either the MagneJector 10 or MagneJector 50 could use a safety switch (not shown) that would disallow the triggering of the needle motion until that safety switch was disengaged.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. A marker delivery device having an elongated permanent magnet of length “2L” placed within a distal section of an elongated hollow needle that is situated at a distal section of the marker delivery device, the marker delivery device having a cylindrical push rod placed within the needle such that the distal end of the rod is in contact with the proximal end of the magnet and the distal end of the magnet is generally situated at the distal end of the needle, the distal tip of the needle being adapted to be placed at a first position which is at the approximate center of tissue within a human subject that is suspected of being cancerous, the marker delivery device then being actuated to advance the needle tip to a second position which advances the distal end of the needle, and the distal end of the rod and also advances the position of the magnet by an approximate distance “L”, the marker delivery device then retracting the needle in a backward direction by the approximate distance “2L” to a third position while the rod and magnet remain fixed in their second position so that the magnet is released from the needle with the center of the magnet being placed at the approximate center of the tissue that is thought to be cancerous.

2. The marker delivery device of claim 1 where the magnet has a center anchor that exerts a frictional force on the interior of the needle prior to release of the magnet into the human tissue and the center anchor opens outwardly after it is ejected from the needle so as to maintain the magnet at the approximate center of the tissue that is suspected of being cancerous.

3. The marker delivery device of claim 1 where the magnet is formed from a permanent magnetic material such as Arnochrome 3, Vicalloy, Alnico 5 or a rare Earth metal.

4. A needle assembly consisting of a needle with a sharpened tip and an elongated permanent magnet placed within a distal portion of that needle, the needle also having a Luer fitting fixedly attached at its proximal end which is removably attached to a Luer fitting that is fixedly attached to the proximal end of an elongated rod situated within the needle with the distal end of the rod being in contact with the proximal end of the elongated permanent magnet, the needle assembly plus elongated rod being adapted for the needle tip to be placed at a first position which is at the approximate center of tissue within a human subject that is suspected of being cancerous.

5. The needle assembly of claim 4 being attached to a MagneJector that is capable of a forward motion of one half the length of the magnet followed by a backward motion of at least the length of the magnet so as to release the magnet into the female breast with the center of the magnet being at the approximate center of the tissue that is to be excised by a surgeon.
